(19) United States
(12) Patent Application Publication
(10) Pub. No.: US 2017/0112444 A1
(43) Pub. Date: Apr. 27, 2017

(54) BIO-SIGNAL SENSOR

(71) Applicant: National Chiao Tung University, Hsinchu (TW)

(72) Inventors: Chin-Teng Lin, Hsinchu (TW); Shao-Wei Lu, Hsinchu (TW); Che-Lun Chang, Hsinchu (TW); Yi-Hsin Yu, Hsinchu (TW)

(21) Appl. No.: 15/297,798

(22) Filed: Oct. 19, 2016

(30) Foreign Application Priority Data
Oct. 21, 2015 (TW) 104134504

(51) Int. Cl. A61B 5/00; A61B 5/1477; A61B 5/145; A61B 5/01; A61B 5/0478

(52) U.S. Cl. CPC A61B 5/6843; A61B 5/01; A61B 5/0478; A61B 5/14542; A61B 5/1477; A61B 5/7278; A61B 5/684; A61B 5/7221; A61B 2562/0209; A61B 2562/046

(57) ABSTRACT

A bio-signal sensor is provided, including: a dry electrode having a plurality of probes and a plurality of contacts correspondingly and electrically connected to the probes, wherein each of the probes senses and transmits an electrical signal to the corresponding contact; and a kit replaceably disposed between the dry electrode and a bio-signal measurement device and having a functional circuit for capturing the electrical signals from the contacts so as to generate a bio-signal and a signal output terminal electrically connected to the functional circuit for transmitting the bio-signal to the bio-signal measurement device.

### BACKGROUND OF THE INVENTION

1. Technical Field

The present invention relates to sensors, and more particularly, to a bio-signal sensor.

2. Description of Related Art

Bio-signals such as electrocardiography (ECG), electromyography (EMG), and electroencephalography (EEG) signals have been widely applied in bio-medical fields. Bio-signal measurement devices generally use dry electrodes to sense the bio-electrical signals. The dry electrodes are made of micro-structural probes, such as micro-electro-mechanical system (MEMS) elements, carbon nanotubes or silver-glass silicones. Generally, such a dry electrode is in direct contact with the skin of a measured portion (for example, the head, torso or limb of a person) for achieving a better sensing effect. However, if the dry electrode is not closely attached to the skin, bio-signals cannot be effectively sensed; otherwise, if the dry electrode is closely attached to the skin, the person may feel uncomfortable.

Further, through a brain-computer interface, bio-signals can be applied in monitoring and control fields for, for example, monitoring a car driver's fatigue state or generating a computer control command. Similarly, portable and real-time monitoring devices use dry electrodes to sense bio-signals and convert the sensed bio-signals into monitoring information or control commands through related circuits or programs.

However, there are some drawbacks in the application of bio-signals in real-time monitoring fields. For example, a wearable EEG measurement device (such as an EEG cap) needs to select a suitable dry electrode according to such factors as different shapes of users' heads, users' motions (for example, shake and sweat) and external environments (for example, temperature, humidity and electromagnetic interference). Further, during replacement of the dry electrode, it takes much time to re-adjust a number of parameters of the EEG cap and position of the dry electrode, thus adversely affecting the real-time monitoring effect.

Therefore, there is a need to provide a bio-signal sensor so as to overcome the above-described drawbacks.

### SUMMARY

The present invention provides a bio-signal sensor, which comprises: a dry electrode comprising a plurality of probes and a plurality of contacts correspondingly and electrically connected to the probes, wherein each of the probes senses an electrical signal of a measured portion of a subject and transmits the electrical signal to the corresponding contact; and a kit replaceably disposed between the dry electrode and a bio-signal measurement device and comprising a functional circuit for capturing the electrical signals from the contacts so as to generate a bio-signal and a signal output terminal electrically connected to the functional circuit for transmitting the bio-signal to the bio-signal measurement device.

According to the bio-signal sensor of the present invention, the kit is replaceably disposed between the dry electrode and the bio-signal measurement device. Therefore, after the dry electrode that is suitable to the shape of a measured portion is determined, kits having different functional circuits can be replaceably disposed between the dry electrode and the bio-signal measurement device so as to provide bio-signal sensors having different functions, thus meeting different application requirements and generating sensitive and accurate bio-signals.

### BRIEF DESCRIPTION OF DRAWINGS

FIGS. 1A and 1B are schematic cross-sectional views of a bio-signal sensor according to a first embodiment of the present invention;

FIGS. 2A and 2B are schematic cross-sectional views of a bio-signal sensor according to a second embodiment of the present invention; and

FIGS. 3A and 3B are schematic cross-sectional views of a bio-signal sensor according to a third embodiment of the present invention.

### DETAILED DESCRIPTION

The following illustrative embodiments are provided to illustrate the present invention, these and other advantages and effects can be apparent to those in the art after reading this specification. It should be noted that all the drawings are not intended to limit the present invention. Various modifications and variations can be made without departing from the spirit of the present invention.

Further, throughout this specification and appended claims, the terms in a singular form may include be in a plural form unless the context indicates otherwise.

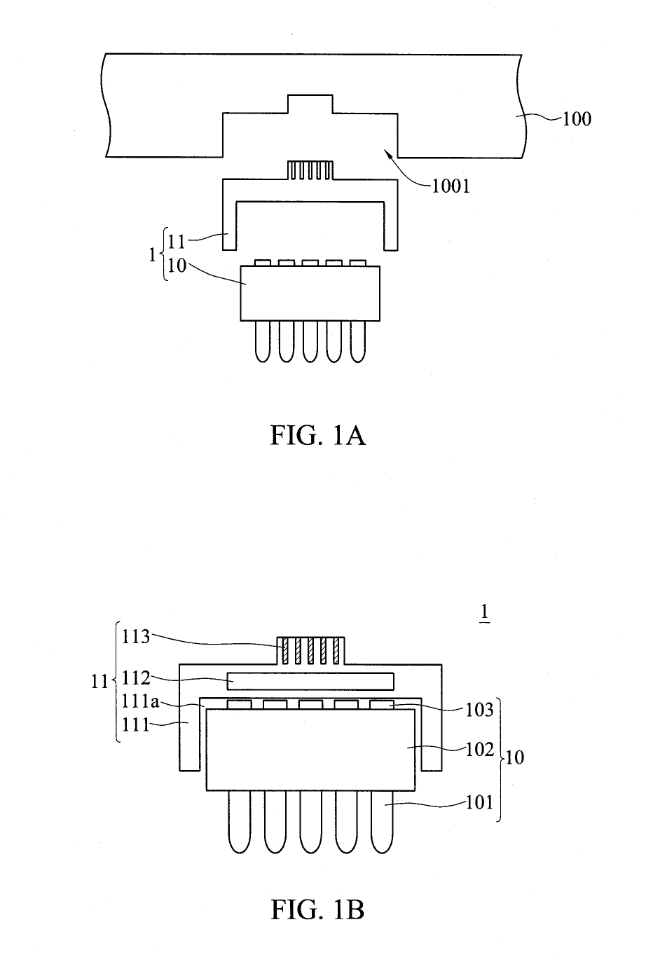

FIGS. 1A and 1B are schematic cross-sectional views of a bio-signal sensor according to a first embodiment of the present invention. Referring to FIG. 1A, a bio-signal measurement device 100 can be provided with a plurality of bio-signal sensors 1 each constituting a bio-signal channel. For example, two to thirty two bio-signal sensors 1 are mounted to the bio-signal measurement device 100. Each of the bio-signal sensors 1 has a dry electrode 10 and a kit 11. The dry electrode 10 is detachably mounted to the kit 11, and the kit 11 is replaceably disposed between the bio-signal measurement device 100 and the dry electrode 10.

Referring to FIG. 1B, the dry electrode 10 has a base 102, and a plurality of probes 101 and a plurality of contacts 103 disposed on two opposite surfaces of the base 102, respectively. The contacts 103 are correspondingly and electrically connected to the probes 101. Each of the probes 101 senses an electrical signal of a measured portion, and transmits the electrical signal to the corresponding contact 103.

The kit 11 has a housing 111 having a receiving space 111a, a functional circuit 112 electrically connected to the contacts 103 for capturing the electrical signals from the contacts 103 so as to generate a bio-signal, and a signal output terminal 113 electrically connected to the functional circuit 112 for transmitting the bio-signal to the bio-signal measurement device 100. The base 102 is detachably received in the receiving space 111a of the housing 111 in a manner that the probes 101 protrude from the receiving space 111a. The signal output terminal 113 is disposed on the housing 111, protruding from a surface of the housing 111.

The base 102 of the dry electrode 10 is made of an insulating material, such as rubber, silicone resin or epoxy resin. Each of the probes 101 and the corresponding contact 103 are electrically connected in a point-to-point manner, and the probes 101 or the contacts 103 are electrically isolated from one another by the base 102. The probes and the corresponding contacts form in pairs, and each pair senses and transmits an electrical signal separately. The probes 101 are made of a biocompatible conductive material, such as Au or AgCl, which have excellent electrical and thermal conductivity.

Further, a guide structure (for example, a slider, a guide slot and a stopper) can be disposed on an inner surface of the housing 111 and an outer surface of the base 102 so as to facilitate assembly and disassembly of the dry electrode 10. A circuit substrate (not shown) having the functional circuit 112 can be provided to electrically connect the contacts 103 and the signal output terminal 113. The signal output terminal 113 can be a gold finger connector, which engages with an insert slot 1001 of the bio-signal measurement device 100, as shown in FIG. 1A.

The functional circuit 112 can include at least one of a microcontroller, an impedance analysis circuit and a parallel circuit, and the kit 11 having the particular functional circuit 112 corresponds to a particular bio-signal generation method. The bio-signal generated by the functional circuit 112 according to the electrical signals can relate to one of the group consisting of EEG, body temperature and oxygen concentration.

Therefore, after the dry electrode 10 that is suitable to the shape of the measured portion is determined, kits 11 having different functional circuits can be replaceably disposed between the dry electrode 10 and the bio-signal measurement device 100 so as to meet different application requirements. For example, since the probes 101 having excellent thermal conductivity can conduct heat of the measured portion to the contacts 103, when measurement is changed from EEG measurement to body temperature measurement, only the kit 11 needs to be replaced with a kit 11 having a temperature measurement function. As such, the functional circuit 112 of the new kit 11 receives heat from the contacts 103 of the dry electrode 10 so as to generate a body temperature signal for the measured portion.

In an embodiment, the functional circuit 112 generates a bio-signal as follows: the functional circuit 112 determines whether the impedance of the electrical signal from each of the contacts 103 is less than a predetermined value, and captures the electrical signal to generate the bio-signal if the impedance of the electrical signal is less than the predetermined value.

In an embodiment, the functional circuit 112 includes a microcontroller and an impedance analysis circuit. When the measurement starts, the impedance analysis circuit sequentially applies a potential difference between two adjacent contacts 103 and analyzes the current therebetween so as to analyze the current impedance between each of the probes 101 and the measured portion. Then, the microcontroller compares the current impedance of the electrical signal from each of the contacts 103 with the predetermined value (a predetermined impedance value, such as 3 KΩ, 5 KΩor 10 KΩ). Subsequently, the electrical signal of the contact 103 with the current impedance less than the predetermined value is captured.

In another embodiment, the functional circuit 12 includes a microcontroller and a pressure analysis circuit. When the measurement starts, the pressure analysis circuit analyzes a pressure value from each of the contacts 103, and compares the pressure value with a predetermined value (e.g., a predetermined pressure value). Subsequently, the electrical signal of the contact 103 with the pressure value less than the predetermined pressure value is captured.

The functional circuit 112, if determining that the current impedance (or the pressure value) of the electrical signal from each of the contacts 103 is greater than the predetermined value (i.e., the contact state between each of the probes 102 and the measured portion does not meet a predetermined criterion), generates a notification signal, and transmits the notification signal through the signal output terminal 113 to the bio-signal measurement device 100, thereby notifying a user (a person who performs the measurement or a subject who is measured) to adjust such as the contact position or tightness degree of the dry electrode 10. Therefore, the user instantly knows which bio-sensor 1 cannot operate normally without the need to check output signals of all the bio-signal sensors 1 of the bio-signal measurement device 100, thereby greatly reducing the adjustment and checking time.

If the captured electrical signal is plural, the functional circuit 112 selects one of the electrical signals that has a least impedance (e.g., a least pressure value or a least current impedance) to generate the bio-signal. The least impedance represents that the corresponding electrical signal sensed by the probe 101 is an optimal one among all the electrical signals By using such a single and optimal electrical signal, the functional circuit 112 prevents noise interference from other probes and hence generates a clearer bio-signal.

In other embodiments, if the captured electrical signal is plural, the functional circuit 112 calculates an average of the electrical signals to generate the bio-signal. As such, the functional circuit 112 generates a more accurate bio-signal.

By electrically connecting the functional circuit 112 to an external device (not shown), the predetermined value can be programmatically set or updated for adjusting the sensitivity of the bio-signal sensor 1.

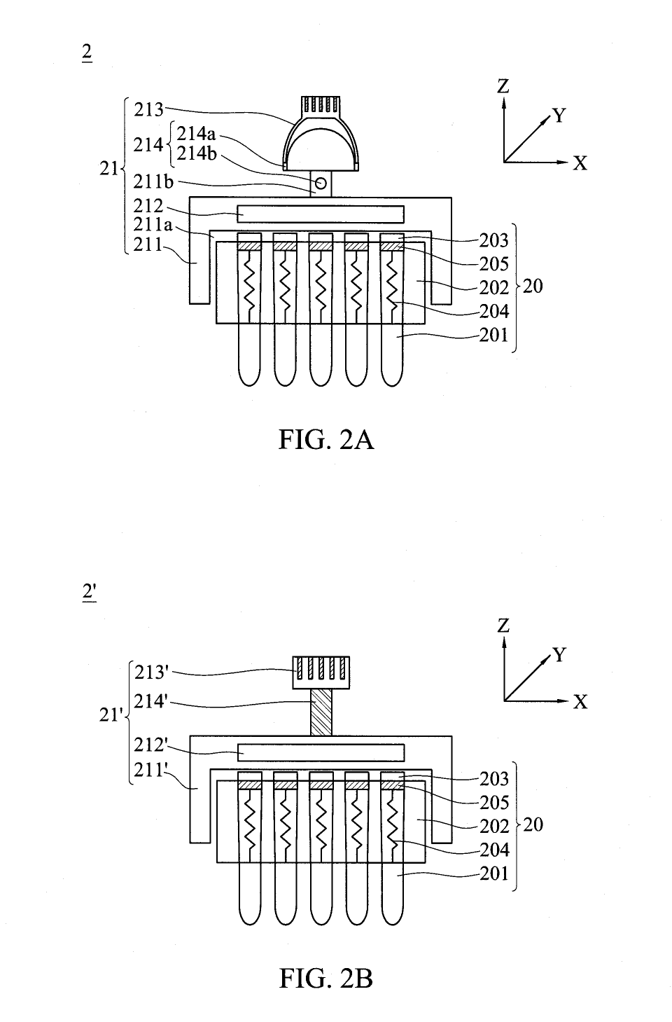

FIG. 2A is a schematic cross-sectional view of a bio-signal sensor 2 according to a second embodiment of the present invention. The bio-signal sensor 2 has a dry electrode 20 and a kit 21.

In addition to a plurality of probes 201, a base 202 and a plurality of contacts 203, the dry electrode 20 has a plurality of elastic conductive elements 204 and a plurality of piezoelectric elements 205. The probes 201 and the contacts 203 are disposed on two opposite surfaces of the base 202, respectively, and each of the probes 201 is electrically connected to the corresponding contact 203 through an elastic conductive element 204 and a piezoelectric element 205. Each of the probes 201 senses an electrical signal of a measured portion and transmits the electrical signal to the corresponding contact 203.

In addition to a housing 211, a functional circuit 212 and a signal output terminal 213, the kit 21 has an adjustment mechanism 214. The base 202 is detachably disposed in the housing 211. The functional circuit 212 is electrically connected to the contacts 203 for capturing electrical signals from the contacts 203 to generate a bio-signal. The adjustment mechanism 214 is connected between the housing 211 and the signal output terminal 213 for adjusting the dry electrode 20, and the signal output terminal 213 is electrically connected through the adjustment mechanism 214 to the functional circuit 212 for transmitting the bio-signal to the bio-signal measurement device 100 (as shown in FIG. 1A).

In an embodiment, the probes 201, the elastic conductive elements 204, the piezoelectric elements 205 and the contacts 103 of the dry electrode 20 are electrically connected in a point-to-point manner. Each of the elastic conductive elements 204 allows the corresponding probe 201 to extend or contract according to a non-planar shape of the measured portion, and the corresponding piezoelectric element 205 receives the deformation force of the elastic conductive element 204 to generate pressure impedance or a pressure value that is used for determining the contact state between the probe 201 and the measured portion.

The adjustment mechanism 214 is disposed between the housing 211 and the signal output terminal 213, two end portions of the signal output terminal 213 are pivotally connected to a rotating shaft 214a of the adjustment mechanism 214 at two sides, and the adjustment mechanism 214 further has a swing shaft 214b passing through and engaged with a protruding ring 211b of the housing 211. After the dry electrode 10 is disposed in the receiving space 211a of the housing 211, the rotating shaft 214a of the adjustment mechanism 214 supports the dry electrode 20 to rotate in a plane Y-Z of FIG. 2A (i.e., rotate out of or into the paper), and the swing shaft 214b of the adjustment mechanism 214 supports the dry electrode 20 to swing in a plane X-Z of FIG. 2A (i.e., parallel to the paper). Therefore, through the adjustment mechanism 214 and the elastic conductive elements 204 of the dry electrode 20, the position of the bio-signal sensor 2 can be adjusted multi-axially (along X, Y and Z axes) according to the three-dimensional shape of the measured portion.

The present invention further provides another type of adjustment mechanism. Referring to FIG. 2B, a kit 21′ of a bio-signal sensor 2′ has a housing 211′, a functional circuit 212′, a signal output terminal 213′ and an adjustment mechanism 214′. The signal output terminal 213′ is electrically connected through the adjustment mechanism 214′ to the functional circuit 212′. The adjustment mechanism 214′ can be a hose such as a shower hose that can rotate freely but cannot be compressed. The adjustment mechanism 214′ supports the dry electrode 20 to rotate in the three-dimensional (XYZ) space of FIG. 2B. Through the adjustment mechanism 214′ and the elastic conductive elements 204, the position of the bio-signal sensor 2′ can be adjusted multi-axially (along X, Y and Z axes) according to the three-dimensional shape of the measured portion.

Therefore, the dry electrode 20 can not only be adapted to a non-planar shape of the measured portion, but also relieve the discomfort of the subject. To meet a different application requirement, for example, when the measurement is changed from EEG or EMG measurement to body temperature or other bio-signal measurement, only the kit 21 or 21′ needs to be changed so as to form a bio-signal sensor 2 or 2′ having the corresponding function.

In the present embodiment, the functional circuit 212 or 212′ generates a bio-signal and/or bio-information as follows: the functional circuit 212 or 212′ determines whether the pressure impedance or the pressure value corresponding to the electrical signal from each of the contacts 203 falls within a predetermined range, and captures the electrical signal to generate the bio-signal and/or the bio-information if the pressure impedance or the pressure value corresponding to the electrical signal falls within the predetermined range.

In an embodiment, the functional circuit 212 or 212′ includes a microcontroller and an impedance analysis circuit. When the measurement starts, the impedance analysis circuit receives the voltage value of each of the piezoelectric elements 205 through the contacts 203 so as to analyze the pressure impedance or the pressure value between each of the probes 201 and the measured portion. Then, the microcontroller compares the pressure impedance or the pressure value of the electrical signal from each of the contacts 203 with a predetermined lower limit value (for example, a predetermined pressure value of 3 Kg/cm²). Subsequently, the electrical signal of the contact 203 with the pressure impedance or the pressure value greater than the predetermined lower limit value is captured.

The microcontroller can compare the pressure impedance or the pressure value of the electrical signal from each of the contacts 203 with a predetermined upper limit value (for example, a predetermined pressure value of 10 Kg/cm²). If the pressure impedance or the pressure value is greater than the predetermined upper limit value, the dry electrode 20 may cause discomfort of the subject due to long-time measurement. Therefore, if the pressure impedance or the pressure value is greater than the predetermined upper limit value, the functional circuit 212 or 212′ generates a notification signal so as to notify the user to adjust the dry electrode 20, thus ensuring the comfort of the subject.

The functional circuit 212 or 212′, if determining that the pressure impedance or the pressure value of the electrical signal from each of the contacts 203 falls out of the predetermined range (for example, the pressure impedance or the pressure value is lower than 3 Kg/cm² or greater than 10 Kg/cm²), generates a notification signal and transmits the notification signal through the signal output terminal 213 or 213′ to the bio-signal measurement device 100 so as to notify the user to adjust the dry electrode 20. Therefore, the user instantly knows which bio-sensor cannot operate normally without the need to check the output signals of all the bio-signal sensors 2 or 2′ of the bio-signal measurement device 100, thereby greatly reducing the adjustment and checking time.

If the captured electrical signal is plural, the functional circuit 212 or 212′ can select one of the electrical signals that has a greatest pressure impedance or pressure value to generate the bio-signal and/or the bio-information. Alternatively, the functional circuit 212 or 212′ further includes a parallel circuit, and if the captured electrical signal is plural, the functional circuit 212 or 212′ guides the captured electrical signals into the parallel circuit and thereby calculates an average of the captured electrical signals to generate the bio-signal and/or the bio-information. As such, the functional circuit 212 or 212′ generates clear and accurate bio-signals and/or the bio-information.

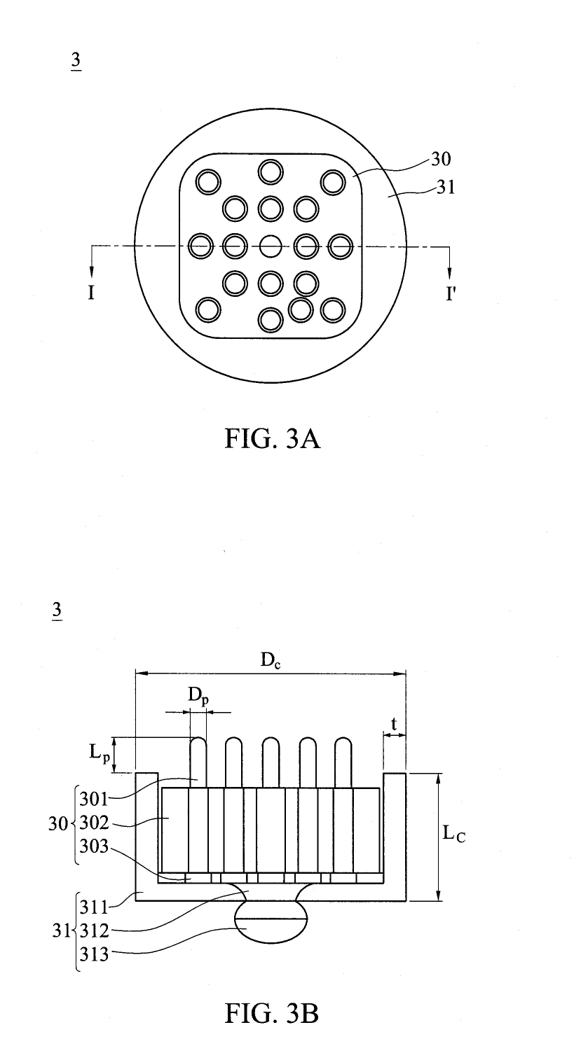

FIG. 3A is a schematic upper view of a bio-signal sensor 3 according to a third embodiment of the present invention. FIG. 3B is a schematic cross-sectional view of the bio-signal sensor 3 taken along a line I-I′ of FIG. 3A. Referring to FIGS. 3A and 3B, the bio-signal sensor 3 has a dry electrode 30 and a kit 31.

The dry electrode 30 has a plurality of probes 301, a base 302 and a plurality of contacts 303. The kit 31 has a housing 311, a functional circuit 312 and a signal output terminal 313.

The configuration of the dry electrode 30 and the kit 31 can be determined according to the practical need. For example, the base 301 can be a prism or cylinder. The probes 302 can be arranged in an array pattern, a star pattern, a ring pattern or a combination thereof. The length Lp of the portion of each of the probes 302 protruding above the kit 31 can be in a range from 1 to 3 mm. The diameter Dp of each of the probes 302 can be in a range from 1 to 3 mm. The height Lc of the housing 311 can be in a range from 5 to 7 mm. The diameter Dc of the housing 311 can be in a range from 10 to 20 mm. The thickness t of the housing 311 can be in a range from 0.5 to 1 mm.

The structure and function of the bio-signal sensor 3 of the third embodiment differ from the above-described embodiments in that the functional circuit 312 substantially includes a parallel circuit.

In an embodiment, the functional circuit 312 calculates an average of the electrical signals from all the contacts 303 to generate the bio-signal and/or the bio-information. Since the circuit configuration is reduced, the size of the dry electrode 30 and the kit 31 can be reduced, thus allowing the bio-signal sensor 3 to have a minimized size without reducing the sensing accuracy. Such a bio-signal sensor 3 can meet the application requirement of small size.

According to the bio-signal sensor of the present invention, the kit is replaceably disposed between the dry electrode and the bio-signal measurement device. Therefore, after the dry electrode that is suitable to the shape of a measured portion is determined, kits having different functional circuits can be replaceably disposed between the dry electrode and the bio-signal measurement device so as to provide bio-signal sensors having different functions, thus meeting different application requirements and generating sensitive and accurate bio-signals and/or the bio-information.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. A bio-signal sensor, comprising:
a dry electrode comprising a plurality of probes and a plurality of contacts correspondingly and electrically connected to the probes, wherein each of the probes senses an electrical signal of a measured portion of a subject and transmits the electrical signal to the corresponding contact; and
a kit replaceably disposed between the dry electrode and a bio-signal measurement device and comprising a functional circuit for capturing the electrical signals from the contacts so as to generate a bio-signal and a signal output terminal electrically connected to the functional circuit for transmitting the bio-signal to the bio-signal measurement device.

2. The bio-signal sensor of claim 1, wherein the dry electrode further comprises a base, and the probes and the corresponding contacts are disposed on two opposite surfaces of the base, respectively.

3. The bio-signal sensor of claim 2, wherein the kit further comprises a housing having a receiving space, the signal output terminal is disposed on the housing, and the base of the dry electrode is replaceably disposed in the receiving space of the housing in a manner that the probes of the dry electrode protrude from the receiving space.

4. The bio-signal sensor of claim 1, wherein the functional circuit comprises at least one of a microcontroller, an impedance analysis circuit and a parallel circuit.

5. The bio-signal sensor of claim 1, wherein the dry electrode further comprises a plurality of elastic conductive elements correspondingly and electrically connected to the probes and a plurality of piezoelectric elements correspondingly and electrically connected between the elastic conductive elements and the contacts.

6. The bio-signal sensor of claim 1, wherein the kit further comprises an adjustment mechanism disposed between the functional circuit and the signal output terminal, and the signal output terminal is electrically connected via the adjustment mechanism to the functional circuit.

7. The bio-signal sensor of claim 1, wherein the bio-signal generated by the functional circuit according to the electrical signals relates to one of the group consisting of EEG, body temperature and oxygen concentration.

8. The bio-signal sensor of claim 1, wherein the functional circuit determines whether current impedance of the electrical signal from each of the contacts is less than a predetermined value, and captures the electrical signal to generate the bio-signal if the current impedance of the electrical signal is less than the predetermined value.

9. The bio-signal sensor of claim 8, wherein the captured electrical signals are plural, and the functional circuit selects one of the captured electrical signals that has a least current impedance to generate the bio-signal.

10. The bio-signal sensor of claim 8, wherein the captured electrical signals are plural, and the functional circuit calculates an average of the captured electrical signals to generate the bio-signal.

11. The bio-signal sensor of claim 8, wherein the functional circuit generates a notification signal and transmits the notification signal through the signal output terminal to the bio-signal measurement device if the current impedance of the electrical signal is greater than the predetermined value, thereby notifying a user to adjust the dry electrode.

12. The bio-signal sensor of claim 8, wherein the predetermined value is programmatically set or updated by electrically connecting the functional circuit to an external device.

13. The bio-signal sensor of claim 1, wherein the functional circuit determines whether pressure impedance or a pressure value of the electrical signal from each of the contacts falls within a predetermined range, and captures the electrical signal to generate the bio-signal if the pressure impedance or the pressure value of the electrical signal falls within the predetermined range.

14. The bio-signal sensor of claim 13, wherein the captured electrical signals are plural, and the functional circuit selects one of the captured electrical signals that has a greatest pressure impedance or pressure value to generate the bio-signal.

15. The bio-signal sensor of claim 13, wherein the captured electrical signals are plural, and the functional circuit calculates an average of the captured electrical signals to generate the bio-signal.

16. The bio-signal sensor of claim 13, wherein the functional circuit generates a notification signal and transmits the notification signal through the signal output terminal to the bio-signal measurement device if the pressure impedance or the pressure value of the electrical signal falls out of the predetermined range, thereby notifying the user to adjust the dry electrode.

17. The bio-signal sensor of claim 13, wherein the predetermined range is programmatically set or updated by electrically connecting the functional circuit to an external device.

18. The bio-signal sensor of claim 1, wherein the functional circuit comprises a microcontroller and a pressure analysis circuit.
